(19) United States
(12) Patent Application Publication
(10) Pub. No.: US 2026/0232911 A1
(43) Pub. Date: Aug. 13, 2026

(54) MEDICAL FILTRATION ASSEMBLY FOR MEDICAL DEVICES

(71) Applicant: Becton, Dickinson and Company, Franklin Lakes, NJ (US)

(72) Inventors: Praveen Nalawade, Belgaum (IN); Shishir Prasad, Ramsey, NJ (US); Ming F. Hsu, Wayne, NJ (US); Tyler Warner, Bluffdale, UT (US); Fanqing Meng, Buffalo Grove, IL (US); Nathan Aaron Mitchell, Murray, UT (US); Adel Shams, Verona, NJ (US); Kristin M. Conway, Martinsville, NJ (US); Jennifer Hooper, Oviedo, FL (US)

(73) Assignee: Becton, Dickinson and Company, Franklin Lakes, NJ (US)

(21) Appl. No.: 19/047,768

(22) Filed: Feb. 7, 2025

(51) Int. Cl. A61M 5/31; A61M 5/178; A61M 5/32

(52) U.S. Cl. CPC A61M 5/3145; A61M 5/1782; A61M 5/3202; A61M 2205/7545

(57) ABSTRACT

A medical filtration assembly configured to be attached to a medical device comprises a tubular adapter comprising a proximal end and a distal end including an opening. The medical filtration assembly includes a filter element on the distal end of the tubular adapter and covering the opening, a needle hub comprising a proximal end and a distal end, the proximal end of the of the tubular adapter configured to cover the distal end of the needle hub, and a removable shield. The removable shield comprises an open proximal end and a distal end extending past the distal end of the tubular adapter, the removable shield sized and configured to cover a portion of the tubular adapter including the distal end of the tubular adapter.

### TECHNICAL FIELD

Embodiments of the disclosure generally relate to integrated filtration assemblies configured for use with medical devices. In particular, embodiments of the disclosure relate to an aspiration and injection assembly with at least one of an integrated filtering mechanism and a safety mechanism for use with syringes in both intravenous (IV) and direct patient injection applications.

### BACKGROUND

Liquid drugs and other pharmaceuticals are typically stored in glass ampoules or vials and sealed with elastomer stoppers, for example, a stopper with a slitted septum. Typically, a two-stage drug administration process is employed to aspirate the drug contained within the vial to a syringe with a fill needle that is subsequently replaced with a hypodermic injection needle prior to administration to a patient. In this typical two-stage process, a blunt fill needle is affixed to a Luer connector of the syringe, to pierce the stopper/septum, followed by retraction of the syringe plunger to aspirate the drug into the syringe chamber. After filling the syringe with a desired quantity of the drug, the blunt fill needle is then detached from the syringe and replaced with a hypodermic injection needle for injection of the drug into the patient.

To administer such medications to a patient, medical professionals must break open the glass ampoule by snapping the glass neck of the ampoule, which may introduce glass shards and other debris into the pharmaceutical product. The glass shards and other debris must be removed from the contaminated pharmaceutical prior to administration to a patient. The glass shards or other debris are typically removed by drawing up the contaminated pharmaceutical through a filtering device secured to the end of the syringe. The filtering device typically comprises a tube, for example, a fill needle, a cannula or a straw that incorporates a filter element. However, this current solution is cumbersome, time-consuming, and involves multiple types of needles to ensure that the medication remains free of glass particulate contamination. Also, after filtration with filter needles, medical professionals must switch to either blunt fill needles (BFN) to transfer the medication from the syringe to an intravenous (IV) bag or IV line (tubing), or to hypodermic needles for direct injection of the medication into a patient. This multi-step process is cumbersome and time-consuming and introduces several hazards, including contamination, needle-stick injuries, and delays in treatment delivery.

Accordingly, there exists an unmet need for a device configured to filter medications from glass ampoules that enhances the safety and efficiency of filtration. Existing methods often carry heightened risks of medication contamination, needle-stick injuries to healthcare providers, and treatment delays, which collectively increase health risks for both patients and medical staff. An improved filtration device would address these issues by reducing needle-stick incidents among healthcare professionals and supporting consistent, high standards of patient care.

### SUMMARY

One or more embodiments of the disclosure are directed to a medical filtration assembly configured to be attached to a medical device. The medical filtration assembly comprises a tubular adapter comprising a proximal end and a distal end including an opening; a filter element on the distal end of the tubular adapter and covering the opening; a needle hub comprising a proximal end and a distal end, the proximal end of the of the tubular adapter configured to cover the distal end of the needle hub; and a removable shield comprising an open proximal end and a distal end extending past the distal end of the tubular adapter, the removable shield sized and configured to cover a portion of the tubular adapter including the distal end of the tubular adapter. In one or more embodiments, the medical filtration assembly may include a blunt fill needle, a hypodermic needle, or a plastic cannula extending from the needle hub. Some embodiments further comprise a safety shield configured to cover a distal end of a needle mounted to the needle hub.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate embodiments of this disclosure and are therefore not to be considered limiting of its scope, for the disclosure may admit to other equally effective embodiments.

FIG. 1A is a partial cross-sectional side view of a first embodiment of a medical filtration assembly configured for use with a medical device and comprising a transparent shield covering a tubular adapter attached to a blunt fill needle hub with the transparent shield in cross-section;

FIG. 1B is a cross-sectional side view of the assembly shown in FIG. 1A, wherein the tubular adapter comprises a filter, and the blunt fill needle hub comprises a needle with the shield and the tubular adapter in cross-section;

FIG. 1C is an expanded side view of the blunt fill needle hub shown in FIG. 1B with a sealing interface between the tubular adapter and the blunt fill needle hub;

FIG. 1D is an exploded isometric view of the medical filtration assembly components shown in FIGS. 1A and 1B;

FIGS. 2A-2D illustrate a method of preparing the medical filtration assembly shown in FIGS. 1A and 1B for IV bag or IV-line medication injection, according to one or more embodiments;

FIG. 3A is a side view of a medical filtration assembly according to one or more embodiments comprising a transparent shield covering a tubular adapter attached to a hypodermic needle hub;

FIG. 3B is a cross-sectional side view of the medical filtration assembly shown in FIG. 3A, wherein the tubular adapter comprises a filter, and the hypodermic needle hub comprises a needle;

FIG. 3C is an expanded side view of the hypodermic needle hub shown in FIG. 3B with a sealing interface between the tubular adapter and the hypodermic needle hub;

FIG. 3D is an exploded isometric view of the medical filtration assembly shown in FIGS. 3A and 3B;

FIGS. 4A-4D illustrate a method of preparing the medical filtration assembly shown in FIGS. 4A-4D for direct medication injection to a patient according to one or more embodiments;

FIG. 5A is an isometric view of a medical filtration assembly according to one or more embodiments including a snap fitting;

FIG. 5B is a cross-sectional side view of the medical filtration assembly shown in FIG. 5A;

FIG. 5C is an expanded side view of the needle hub shown in FIG. 5B, with a sealing interface between the tubular adapter and the needle hub;

FIG. 6A is an isometric view of a medical filtration assembly according to one or more embodiments, including a washer;

FIG. 6B is a cross-sectional side view of the medical filtration assembly shown in FIG. 5A without the shield;

FIG. 6C is an expanded side view of the filter and adapter interface shown in FIG. 6B;

FIG. 7A is an isometric view of another embodiment of a medical filtration assembly including an extruded adapter and shield;

FIG. 7B is a cross-sectional side view of the assembly shown in FIG. 7A;

FIG. 8A is an isometric view of a medical filtration assembly according to another embodiment;

FIG. 8B is a cross-sectional side view of the medical filtration assembly shown in FIG. 8A;

FIGS. 9A-9D illustrate a method of using the medical filtration assembly shown in FIGS. 8A and 8B for direct medication injection to a patient;

FIG. 10A is a side view of a medical filtration assembly according to another embodiment;

FIG. 10B is a cross-sectional side view of the medical filtration assembly shown in FIG. 10A;

FIG. 10C is an expanded side view of the plastic needle hub shown in FIG. 10B, showing a sealing interface between the tubular adapter and the plastic needle hub;

FIG. 10D is an exploded isometric view of the medical filtration assembly shown in FIGS. 10A and 10B;

FIGS. 11A-11D illustrate a method of preparing the medical filtration assembly shown in FIGS. 10A and 10B for direct medication injection to a patient;

FIG. 12A is an isometric view of a medical filtration assembly including a hinged safety shield according to another embodiment;

FIG. 12B is a cross-sectional side view of the medical filtration assembly shown FIGS. 12A and 12B;

FIGS. 12C-12E are expanded side views of the sealing interference interaction between the tubular adapter and needle hub interface shown in FIG. 12B;

FIGS. 13A-13F illustrate a method of preparing the medical filtration assembly shown in FIG. 12A for direct medication injection to a patient;

FIG. 14A is an isometric of view of a medical filtration assembly including a sliding safety shield according to another embodiment;

FIG. 14B is a cross-sectional side view of the medical filtration assembly shown in FIG. 14A;

FIGS. 14C and 14D are expanded side views of the sealing interference interaction between the tubular adapter and needle hub interface of the medical filtration assembly shown in FIG. 14B;

FIG. 15A is an isometric view of a medical filtration assembly with a push type safety shield according to another embodiment;

FIG. 15B is a cross-sectional side view of the medical filtration assembly shown in FIG. 15A;

FIGS. 15C and 15D are expanded side views of the sealing interference interaction between the tubular adapter, syringe, and safety shield interface shown in FIG. 14B;

FIG. 16A is an isometric view of a medical filtration assembly including a sliding safety shield according to another embodiment;

FIG. 16B is a cross-sectional side view of the medical filtration assembly shown in FIG. 16A;

FIG. 16C is a side view of the medical filtration assembly shown in FIG. 16A before activation of the sliding safety shield;

FIG. 16D is a side view of the medical filtration assembly shown in FIG. 16A after removal of the shield and adapter and activation of the sliding safety shield to cover exposed needle;

FIG. 17A is an isometric view of a medical filtration assembly including a passive safety mechanism according to another embodiment;

FIG. 17B is a side view of the medical filtration assembly shown in FIG. 17A before activation of a spring-loaded safety shield; and

FIG. 17C is a side view of the medical filtration assembly shown in FIG. 17A after removal of the shield and adapter, and the activation of the spring-loaded safety shield.

### DETAILED DESCRIPTION

In this disclosure, a convention is followed wherein the distal end of the medical filtration assembly is the end closest to a patient, e.g., for delivery of one or more drugs to the patient, and the proximal end of the medical filtration assembly is the end away from the patient and closest to a clinician or other medical practitioner. With respect to terms used in this disclosure, the following definitions are provided.

As used herein, the use of “a,” “an,” and “the” includes the singular and plural.

As used herein, the term “Luer connector” refers to a connection collar that is the standard way of attaching syringes, catheters, hubbed needles, I.V. tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is associated with a syringe discharge tip and can interlock and connect to the female end that is incorporated within a proximal end of a needle hub of a drug dispensing needle or filling needle for aspirating a drug from a drug vial into a syringe. Luer connector female ends are commonly located on vascular access devices (VADs).

As used herein, ISO 80369-7:2016 defines a specification for standard Luer connectors including a 6% taper between the distal end and the proximal end. A male standard Luer connector increases from the open distal end to the proximal end. A female standard Luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard Luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “wall”, “top”, “side”, “bottom” and others are used throughout this specification to facilitate understanding, it is not intended to limit any components that can be used in combinations or individually or to require specific spatial orientations, to implement various aspects of the embodiments of the present disclosure.

According to one or more embodiments, when an element or structure is referred to as being “configured to” perform a particular function, or “made to” or “designed to” perform that function, however, when the disclosure makes clear that the recited structure is “designed to” or “constructed to” perform that function, the element or structure is designed, made or configured to accomplish the specific objective.

Before describing several exemplary embodiments of the disclosure, it is to be understood that the disclosure is not limited to the details of construction or process steps set forth in the following description. The disclosure is capable of other embodiments and of being practiced or being carried out in various ways. According to one or more embodiments, a “filter element” refers to a porous element configured to filter particles from a liquid medium. In one or more embodiments, the filter element is constructed of porous polymers, such as an elastomer having a Shore D hardness in a range of 30-70. In one or more embodiments, the filter element is optimized and configured to filter particles, in particular, glass particles that have a size greater than 5 μm from a liquid medium. In some embodiments, the filter element is fabricated by blowing out air or blowing agent during injection or hot press molding (such as microcellular injection molding). In other embodiments, the filter element is fabricated by preferential solubilization or precipitation of mixed particles, such as salt. In yet other embodiments, the filter element is fabricated by thermal, laser or ultrasonic welding or sintering of polymeric material. Exemplary polymeric materials include but are not limited to polypropylene, polyethylene, polystyrene, polytetrafluoroethylene (PTFE), acrylic, polyethersulfone, Nylon, Cellulose Nitrate.

One or more embodiments of the disclosure pertain to a medical filtration assembly configured for use with and attachment to a medical device so that the medical device is configured to transfer filtered medication from a container for delivery to a patient. In various embodiments, the medical filtration assembly comprises a tubular adapter comprising a proximal end and a distal end including an opening; a filter element on the distal end of the tubular adapter and covering the opening; a needle hub comprising a proximal end and a distal end, the proximal end of the of the tubular adapter configured to cover the distal end of the needle hub; and a removable shield comprising an open proximal end and a distal end extending past the distal end of the tubular adapter, the removable shield sized and configured to cover a portion of the tubular adapter including the distal end of the tubular adapter. The phrase “needle hub” refers to a hub that may or may not include a needle extending therefrom. In one or more embodiments, the needle hub has a cannula extending therefrom. The cannula may be a blunt fill needle cannula, a hypodermic needle cannula, a plastic tube or a plastic needle as described further herein. The filter element as described herein according to one or more embodiments is configured to filter particles from a liquid medium. One or more embodiments include a medical filtration assembly that is configured to be attached to medical device such as a syringe. Connection can be made by a Luer lock fitting where one component (e.g., the medical filtration assembly) has lugs or external threads and the other component (e.g., the syringe) has a collar with internal threads that engage the lugs or external threads. As used herein, “integrated” refers to an assembly as described further below in which the filter element is integrated with the assembly or made integral with a portion of the assembly.

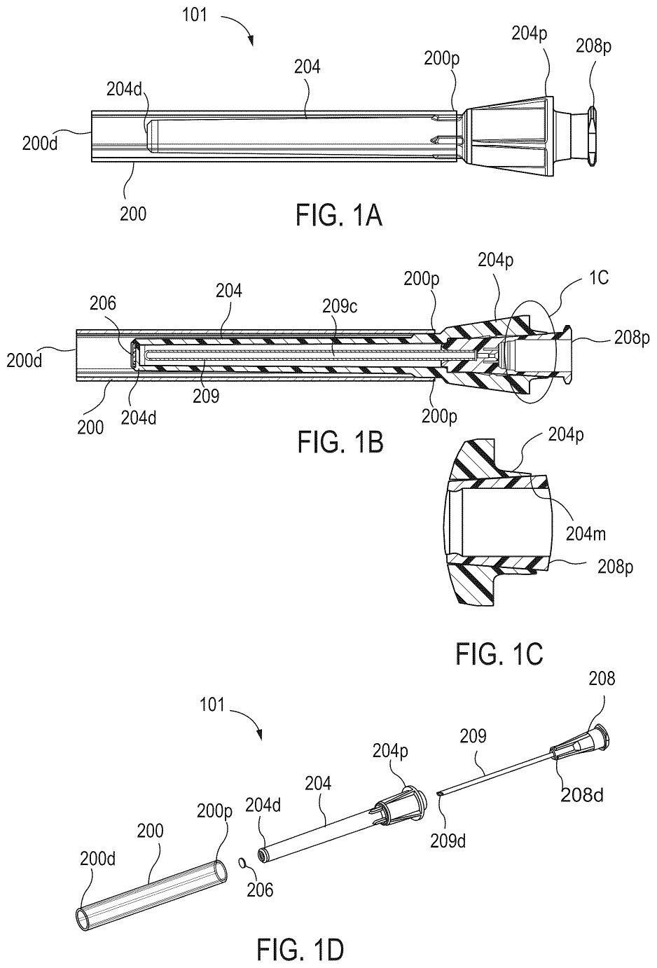

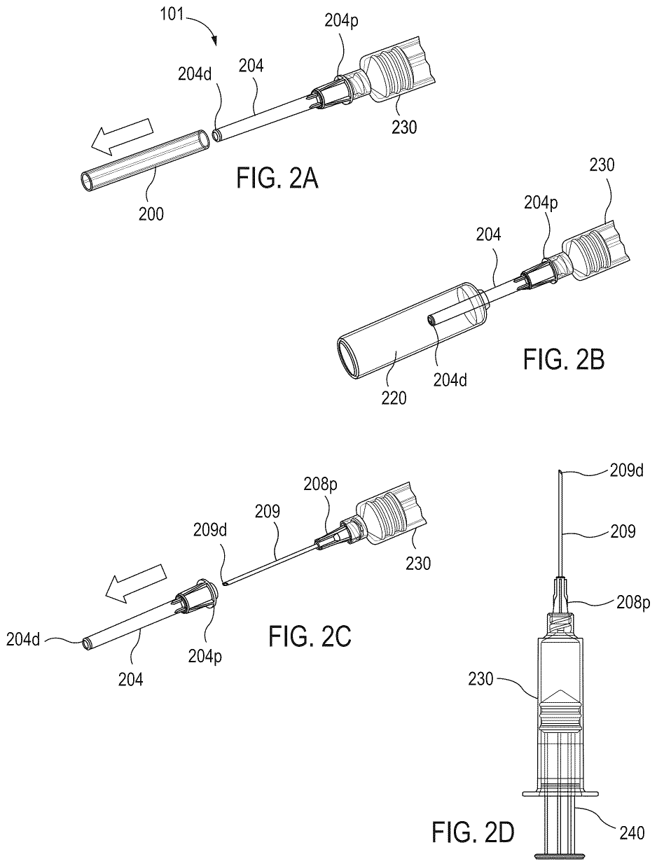

FIGS. 1A-1D show a first embodiment of a medical filtration assembly 101 configured for use with a medical device and configured to transfer filtered medication from a container for delivery to a patient. The medical filtration assembly 101 shown in FIGS. 1A-D is configured to be attached to a medical device such as a syringe 230 (shown in FIGS. 2A-D). The medical filtration assembly 101 is configured so that a syringe 230 attached to the medical filtration assembly 101 is configured to aspirate medication from a glass ampoule 220 and injected into an IV bag (not shown). The medical filtration assembly 101 shown comprises a tubular adapter 204 having a proximal end 204p and a distal end 204d, and each of the proximal end 204p and the distal end 204d of the tubular adapter 204 are open. In one or more embodiments, the tubular adapter 204 comprises plastic material. In some embodiments, the plastic material comprises polypropylene, polyethylene, polycarbonate, or any other suitable plastic material used in the art of medical devices. In one or more embodiments, the tubular adapter 204 fully or partially comprises elastomeric material. In some embodiments, the elastomeric material comprises thermoplastic elastomer (TPE). The tubular adapter comprises a proximal end 204p and a distal end 204d. In some embodiments, the tubular adapter 204 is transparent.

In one or more embodiments, the medical filtration assembly further comprises a needle hub 208 including a distal end 208d and proximal end 208p, and there is a mating surface 204m of the adapter with the needle hub 208 at the proximal end 208p. In some embodiments, the needle hub 208 comprises plastic. In the embodiment shown, the needle hub 208 is a blunt fill needle hub is configured to receive a blunt fill needle, and a blunt fill needle 209 extends from the distal end 208d of the needle hub 208. The blunt fill needle 209 includes a cannula 209c and may be made of metal and have a blunt distal tip 209d. In other embodiments, the blunt fill needle 209 is made of plastic. The blunt fill needle 209 may have a single bevel, have a pencil tip with side port, or any desired tip geometry.

As used herein, a blunt fill needle 209 is distinguished from a hypodermic needle having a sharp distal tip configured to pierce a patient's skin with minimal to no pain. Blunt fill needles 209 have a distal tip 209d that is less sharp than a hypodermic needle distal tip. Blunt fill needles are used to pierce a septum of a vial or a medication bag such as an intravenous medical bag. In some embodiments, the needle hub 208 is attached to the medical filtration assembly 101 by inserting the distal end 208d of the needle hub 208 through the proximal end 204p of the tubular adapter 204. In one or more embodiments, the tubular adapter 204 and the needle hub 208 are assembled together by an interference fit. In some embodiments, the interference fit achieves an airtight seal through plastic-to-plastic contact between the tubular adapter 204 and the needle hub 208. In some embodiments, the tubular adapter 204 is configured as a cover to cover the needle attached to the needle hub.

In one or more embodiments, the distal end 204d of the tubular adapter 204, which is open, comprises a filter element 206. The tubular adapter 204 with the open distal end 204d and the filter element 206 is configured to aspirate medication through the filter element 206. In some embodiments, the filter element 206 is assembled inside the distal end 204d of the tubular adapter 204. In some embodiments the filter element 206 is sealed inside the distal end 204d of the tubular adapter 204 by a sealing method, for example, insert molding, adhesive bonding, ultrasonic welding, or by any other suitable sealing method. In this way, the filter element 206 is integrated with the medical filtration assembly 101. In some embodiments, the filter element 206 has a porosity configured to filter glass particles or glass shards that are present inside a glass ampoule.

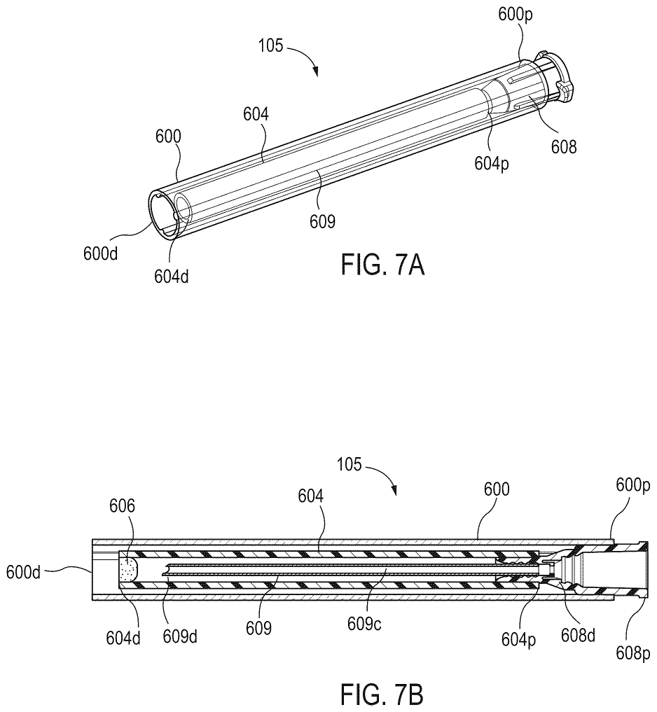

In one or more embodiments, the medical filtration assembly 101 further comprises a shield 200, which is removable and has distal end 200d and a proximal end 200p. In some embodiments, the distal end 200d of the shield 200 is flat and blunt. In other embodiments, the distal end 200d of the shield 200 is closed. In some embodiments, the shield 200 covers the distal end 204d of the tubular adapter 204. In some embodiments, the shield 200 covers a portion of the needle hub 208. In some embodiments, the shield 200 does not cover the needle hub 208, for example, the embodiment shown in FIG. 7. In some embodiments, the shield 200 is transparent. The shield 200 provides protection from foreign material entering the medical filtration assembly prior to its use. In the embodiments shown, the proximal end of the shield 200 is slidably engaged with the tubular adapter 204 to secure the shield 200 to the medical filtration assembly 101.

FIGS. 2A-D illustrate a method of assembling the medical filtration assembly 101 on a syringe 230 to facilitate medication delivery into an IV bag (not shown). FIG. 2A shows that after breaking open a glass ampoule 220 containing medication and removing the medical filtration assembly from its packaging, sliding the shield 200 in the direction of the arrow in FIG. 2A away from the tubular adapter 204 to remove the shield 200 covering the tubular adapter 204 of the medical filtration assembly 101 to expose the tubular adapter 204. As shown in FIG. 2B, the distal end 204d of the tubular adapter 204 is inserted into the ampoule 220, and the medication is aspirated through the tubular adapter 204 of the medical filtration assembly 101 into the syringe 230 attached to the medical filtration assembly 101. In some embodiments, after aspiration of the medication to the syringe 230, the medical filtration assembly 101 with the syringe attached is removed from the ampoule and the tubular adapter is detached from device. In some embodiments, the needle is left attached to the syringe. In some embodiments, the syringe is held vertically to expel air before injecting the medication into an IV bag (not shown).

FIG. 2C illustrates the tubular adapter 204 being removed from the needle hub 208 that has been attached to the syringe 230 by moving the tubular adapter 204 in a distal direction as shown by the hollow arrow in FIG. 2C to expose the distal tip 209d of the blunt fill needle 209. FIG. 2D illustrates the syringe 230 including a plunger rod 240 with needle hub 208 attached held vertically to air from the syringe 230 before delivering the medication in the syringe 230 aspirated from the ampoule 220 to an IV bag (not shown).

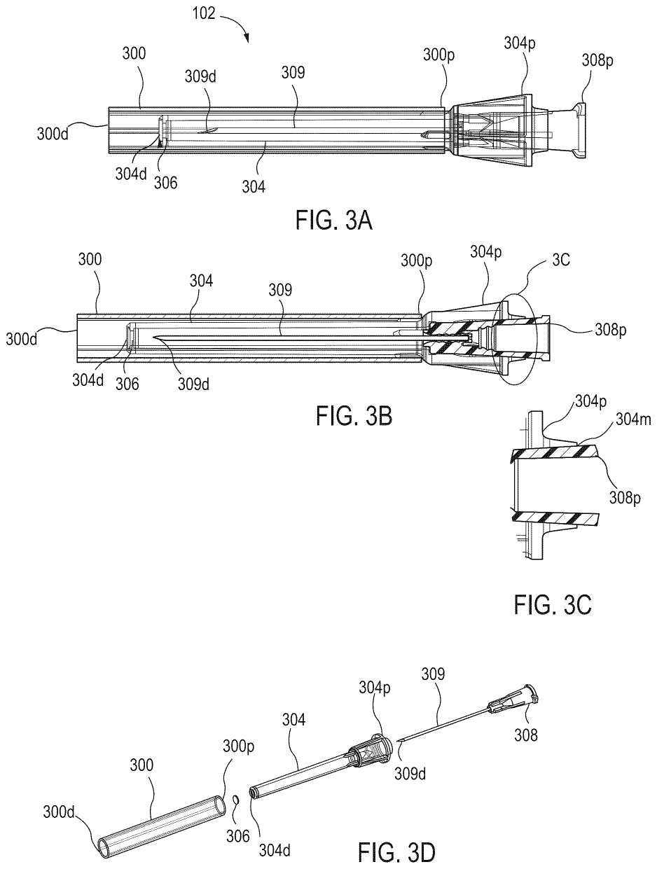

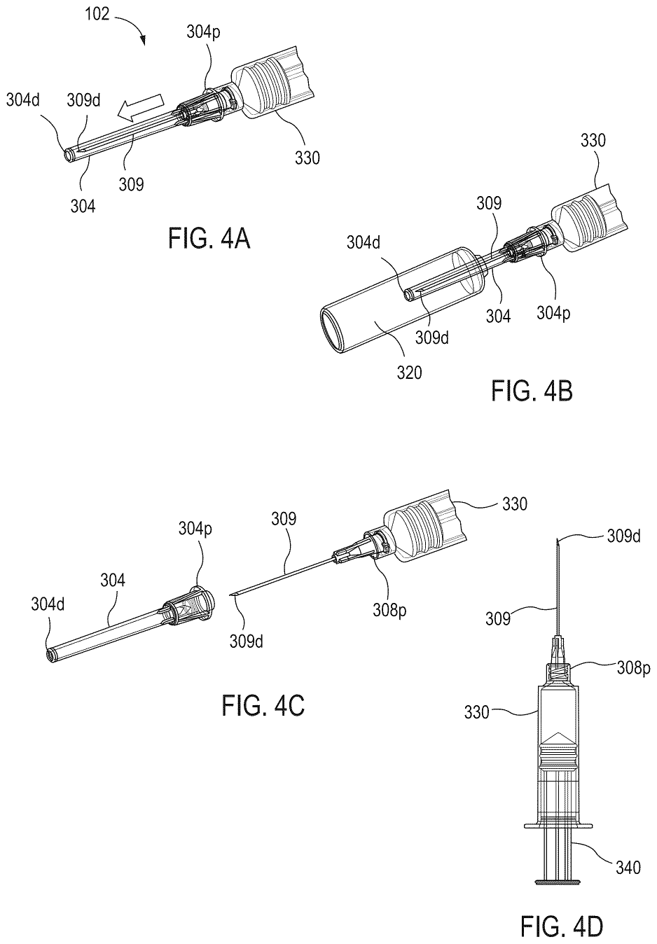

FIGS. 3A-D show an embodiment of a medical filtration assembly 102 configured for use with a medical device and configured to transfer filtered medication from a container for delivery to a patient including a hypodermic needle 309 having a distal tip 309d. The medical filtration assembly 102 shown in FIGS. 3A-D is configured to be attached to a medical device such as a syringe 330 (shown in FIGS. 4A-D). The medical filtration assembly 102 is configured so that a syringe 330 attached to the medical filtration assembly 102 is configured to aspirate medication from a glass ampoule 320 and injected directly to a patient, for example, intravenously.

The medical filtration assembly 102 shown in FIGS. 3A-D is configured to be attached to a medical device such as a syringe 330 (shown in FIGS. 4A-D). The medical filtration assembly 102 is configured so that a syringe 330 attached to the medical filtration assembly 102 is configured to aspirate medication from a glass ampoule 320 and injected directly into a patient. The medical filtration assembly 102 shown comprises a tubular adapter 304 having a proximal end 304p and a distal end 304d, and each of the proximal end 304p and the distal end 304d of the tubular adapter 304 are open. In one or more embodiments, the tubular adapter 304 comprises plastic material. In some embodiments, the plastic material comprises polypropylene, polyethylene, polycarbonate, or any other suitable plastic material used in the manufacture of medical devices. In one or more embodiments, the tubular adapter 304 fully or partially comprises elastomeric material. In some embodiments, the elastomeric material comprises thermoplastic elastomer (TPE). The tubular adapter comprises a proximal end 304p and a distal end 304d. In some embodiments, the tubular adapter 304 is transparent.

In one or more embodiments, the medical filtration assembly further comprises a needle hub 308 including a distal end 308d and proximal end 308p. In some embodiments, the needle hub 308 comprises plastic. In the embodiment shown, the needle hub 308 is a hypodermic needle hub is configured to receive a hypodermic needle, and a hypodermic needle 309 extends from the distal end 308d of the needle hub 308. The hypodermic needle 309 includes a cannula 309c and may be made of metal and have a sharp distal tip 309d configured to pierce a patient's skin for delivery of medication.

In some embodiments, the needle hub 308 is attached to the medical filtration assembly 102 by inserting the distal end 308d of the needle hub 308 through the proximal end 304p of the tubular adapter 304. In one or more embodiments, the tubular adapter 304 and the needle hub 308 are assembled together by an interference fit shown in detail in FIG. 3C, showing the mating surface 304m of the adapter with the needle hub 308. In some embodiments, the interference fit achieves an airtight seal through plastic-to-plastic contact between the tubular adapter 304 and the needle hub 308. In some embodiments, the tubular adapter 304 is configured as a cover to cover the hypodermic needle 309 attached to the needle hub 308.

In one or more embodiments, the distal end 304d of the tubular adapter 304, which is open, comprises a filter element 306. The tubular adapter 304 with the open distal end 304d and the filter element 306 is configured to aspirate medication through the filter element 306. In some embodiments, the filter element 306 is assembled inside the distal end 304d of the tubular adapter 304. In some embodiments the filter element 306 is sealed inside the distal end 304d of the tubular adapter 304 by a sealing method, for example insert molding, adhesive bonding, ultrasonic welding, or by any other suitable sealing method. In this way, the filter element 306 is integrated with the medical filtration assembly 102. In some embodiments, the filter element 306 has a porosity configured to filter glass particles or glass shards that are present inside a glass ampoule.

The medical filtration assembly 102 further comprises a shield 300, which is removable and has a distal end 300d and a proximal end 300p. In some embodiments, the distal end 300d of the shield 300 is flat and blunt. In other embodiments, the distal end 300d of the shield 300 is closed. In some embodiments, the shield 300 covers the distal end 304d of the tubular adapter 304. In some embodiments, the shield 300 covers a portion of the needle hub 308. In some embodiments, the shield 300 does not cover the needle hub 308. In some embodiments, the shield 300 is transparent. The shield 300 provides protection from foreign material entering the medical filtration assembly prior to its use.

FIGS. 4A-D illustrate a method of assembling the medical filtration assembly 102 on a syringe 330 to facilitate medication delivery directly into a patient (not shown). FIG. 4A shows that after breaking open a glass ampoule 320 containing medication and removing the medical filtration assembly from its packaging and after sliding the shield 300 away from the tubular adapter 304 to remove the shield 300 covering the tubular adapter 304 of the medical filtration assembly 102 to expose the tubular adapter 304.

As shown in FIG. 4B, the distal end 304d of the tubular adapter 304 is inserted into the ampoule 320, and the medication is aspirated through the tubular adapter 304 of the medical filtration assembly 102 into the syringe 330 attached to the medical filtration assembly 102. FIG. 4C illustrates the tubular adapter 304 being removed from the needle hub 308 that has been attached to the syringe 330 by moving the tubular adapter 304 in a distal direction as shown by the hollow arrow in FIG. 4C to expose the distal tip 309d of the hypodermic needle 309. FIG. 4D illustrates the syringe 330 including a plunger rod 340 with the needle hub 308 attached held vertically to remove air from the syringe 330 before delivering the medication in the syringe aspirated from the ampoule 320 directly to a patient, for example intravenously. (not shown).

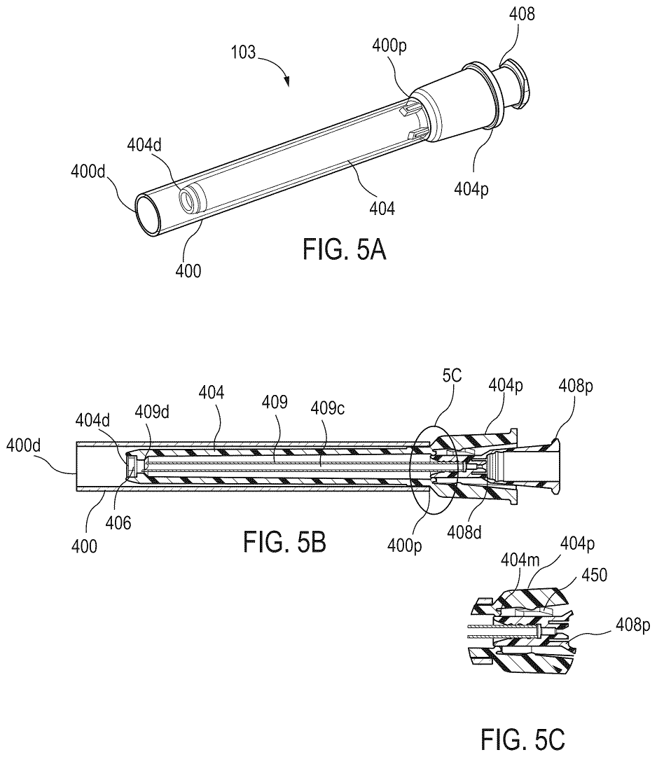

FIGS. 5A-C illustrate third embodiment of a medical filtration assembly 103 including a tubular adapter 404 having a first snap fitting element 450 that engages the needle hub 408.

FIGS. 5A-C show the medical filtration assembly 103 configured for use with a medical device and configured to transfer filtered medication from a container for delivery to a patient or an IV bag including a needle 409 having a distal tip 409d. The medical filtration assembly 103 shown in FIGS. 5A-C is configured to be attached to a medical device such as a syringe as shown in the first two embodiments. The medical filtration assembly 103 is configured so that a syringe (not shown) attached to the medical filtration assembly 103 is configured to aspirate medication from a glass ampoule (not shown) and injected either directly to a patient, for example, intravenously or to an IV bag.

The medical filtration assembly 103 shown in FIGS. 5A-C is configured to be attached to a medical device such as a syringe. The medical filtration assembly 103 is configured so that a syringe attached to the medical filtration assembly 103 is configured to aspirate medication from a glass ampoule and injected directly into a patient or into an IV bag. The medical filtration assembly 103 shown comprises a tubular adapter 404 having a proximal end 404p and a distal end 404d, and each of the proximal end 404p and the distal end 404d of the tubular adapter 404 are open. In one or more embodiments, the tubular adapter 404 comprises plastic material as described above. The tubular adapter comprises a proximal end 404p and a distal end 404d. In some embodiments, the tubular adapter 404 is transparent.

In one or more embodiments, the medical filtration assembly 103 further comprises a needle hub 408 including a distal end 408d and proximal end 408p. In some embodiments, the needle hub 408 comprises plastic. In the embodiment shown, the needle hub 408 is a needle hub is configured to receive a needle 409 which extends from the distal end 408d of the needle hub 408. The needle 409 includes a cannula 409c and may be made of metal and have a distal tip 409d.

In some embodiments, the needle hub 408 is attached to the medical filtration assembly 103 by inserting the distal end 408d of the needle hub 408 through the proximal end 404p of the tubular adapter 404. In one or more embodiments, the tubular adapter 404 and the needle hub 408 are assembled together by a snap fit shown in detail in FIG. 5C, showing the mating surface 404m of the tubular adapter with the needle hub 408. The snap fit achieves secure attachment of the tubular adapter 404 to the needle hub 408. A snap fitting element 450 comprises an elongate finger that engaged a protrusion on the proximal end 404p of the tubular adapter 404.

In one or more embodiments, the distal end 404d of the tubular adapter 404, which is open, comprises a filter element 406. The tubular adapter 404 with the open distal end 404d and the filter element 406 is configured to aspirate medication through the filter element 406. In some embodiments, the filter element 406 is assembled inside the distal end 404d of the tubular adapter 404. In some embodiments the filter element 406 is sealed inside the distal end 404d of the tubular adapter 404 by a sealing method, for example insert molding, adhesive bonding, ultrasonic welding, or by any other suitable sealing method. In this way, the filter element 406 is integrated with the medical filtration assembly 103. In some embodiments, the filter element 406 has a porosity configured to filter glass particles or glass shards that are present inside a glass ampoule.

The medical filtration assembly 103 further comprises a shield 400, which is removable and has a distal end 400d and a proximal end 400p. In some embodiments, the distal end 400d of the shield 400 is flat and blunt. In other embodiments, the distal end 400d of the shield 400 is closed. In some embodiments, the shield 400 covers the distal end 404d of the tubular adapter 404. In some embodiments, the shield 400 covers a portion of the needle hub 408. In some embodiments, the shield 400 does not cover the needle hub 408. In some embodiments, the shield 400 is transparent. The shield 400 provides protection from foreign material entering the medical filtration assembly prior to its use.

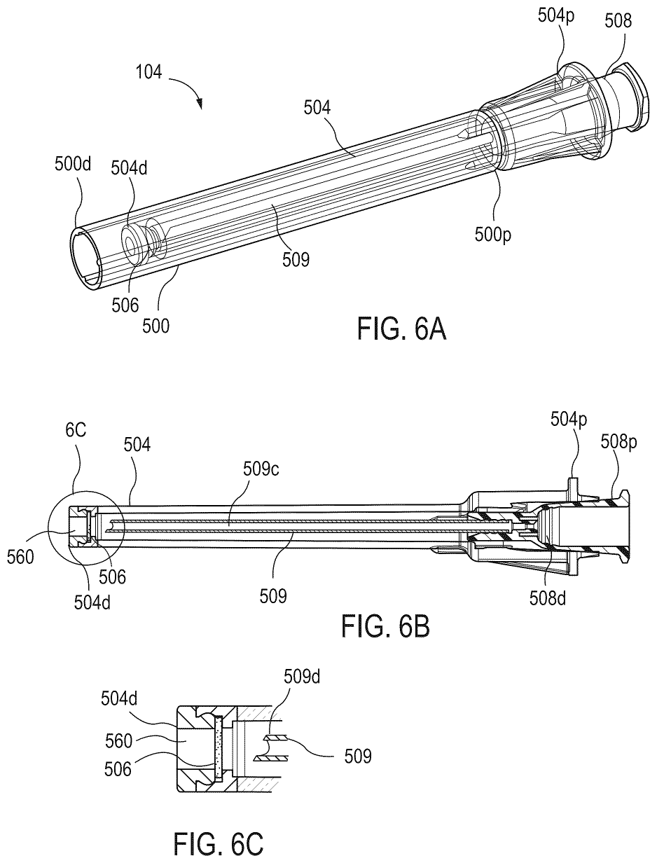

Referring now to FIGS. 6A-C a fourth embodiment of a medical filtration assembly 104 further comprises a washer 560 which holds a filter element 506 in place by press fitting the washer 560 against the filter element 506. The washer 560 is located on the distal end 504d of the tubular adapter 504. In some embodiments, the washer 560 is press-fitted or adhesively bonded to the distal end 504d of the tubular adapter 504. The washer 560 fitted to the distal end of the tubular adapter 504 is in contact with the filter element 506 to secure the filter element 506 to the tubular adapter 504.

FIGS. 6A-C show the medical filtration assembly 104 configured for use with a medical device and configured to transfer filtered medication from a container for delivery to a patient or an IV bag including a needle 509 having a distal tip 509d. The medical filtration assembly 104 shown in FIGS. 6A-C is configured to be attached to a medical device such as a syringe as shown in the first two embodiments. The medical filtration assembly 104 is configured so that a syringe (not shown) attached to the medical filtration assembly 104 is configured to aspirate medication from a glass ampoule (not shown) and injected either directly to a patient, for example, intravenously or to an IV bag.

The medical filtration assembly 104 shown in FIGS. 6A-C is configured to be attached to a medical device such as a syringe. The medical filtration assembly 104 is configured so that a syringe attached to the medical filtration assembly 104 is configured to aspirate medication from a glass ampoule and injected directly into a patient or into an IV bag. The medical filtration assembly 104 shown comprises a tubular adapter 504 having a proximal end 504p and a distal end 504d, and each of the proximal end 504p and the distal end 504d of the tubular adapter 504 are open. In one or more embodiments, the tubular adapter 504 comprises plastic material as described above. The tubular adapter comprises a proximal end 504p and a distal end 504d. In some embodiments, the tubular adapter 504 is transparent.

In one or more embodiments, the medical filtration assembly 104 further comprises a needle hub 508 including a distal end 508d and proximal end 508p. In some embodiments, the needle hub 508 comprises plastic. In the embodiment shown, the needle hub 508 is a needle hub is configured to receive a needle 509 which extends from the distal end 508d of the needle hub 508. The needle 509 includes a cannula 509c and may be made of metal and have a distal tip 509d.

In some embodiments, the needle hub 508 is attached to the medical filtration assembly 104 by inserting the distal end 508d of the needle hub 508 through the proximal end 504p of the tubular adapter 504. In one or more embodiments, the tubular adapter 504 and the needle hub 508 are assembled together by either press fitting or a snap fitting as described above.

In one or more embodiments, the distal end 504d of the tubular adapter 504, which is open, comprises a filter element 506 held in place by the washer 560. The tubular adapter 504 with the open distal end 504d and the filter element 506 is configured to aspirate medication through the filter element 506. In some embodiments, the filter element 506 is assembled inside the distal end 504d of the tubular adapter 504. In some embodiments the filter element 506 is sealed inside the distal end 504d of the tubular adapter 504 by a sealing method, for example insert molding, adhesive bonding, ultrasonic welding, or by any other suitable sealing method. In this way, the filter element 506 is integrated with the medical filtration assembly 104. In some embodiments, the filter element 506 has a porosity configured to filter glass particles or glass shards that are present inside a glass ampoule.

The medical filtration assembly 104 further comprises a shield 500, which is removable and has a distal end 500d and a proximal end 500p. In some embodiments, the distal end 500d of the shield 500 is flat and blunt. In other embodiments, the distal end 500d of the shield 500 is closed. In some embodiments, the shield 500 covers the distal end 504d of the tubular adapter 504. In some embodiments, the shield 500 covers a portion of the needle hub 508. In some embodiments, the shield 500 does not cover the needle hub 508. In some embodiments, the shield 500 is transparent. The shield 500 provides protection from foreign material entering the medical filtration assembly prior to its use.

In a fifth embodiment of a medical filtration assembly 105, shown in FIGS. 7A-B, the proximal end 600p of the removable shield 600 is slidably engaged with the needle hub 608 to secure the removable shield to the medical filtration assembly 105.

FIGS. 7A-B show the medical filtration assembly 105 configured for use with a medical device and configured to transfer filtered medication from a container for delivery to a patient or an IV bag including a needle 609 having a distal tip 609d. The medical filtration assembly 105 shown in FIGS. 7A-B is configured to be attached to a medical device such as a syringe as shown in the first two embodiments. The medical filtration assembly 105 is configured so that a syringe (not shown) attached to the medical filtration assembly 105 is configured to aspirate medication from a glass ampoule (not shown) and injected either directly to a patient, for example, intravenously or to an IV bag.

The medical filtration assembly 105 shown in FIGS. 7A-B is configured to be attached to a medical device such as a syringe. The medical filtration assembly 105 is configured so that a syringe attached to the medical filtration assembly 105 is configured to aspirate medication from a glass ampoule and injected directly into a patient or into an IV bag. The medical filtration assembly 105 shown comprises a tubular adapter 604 having a proximal end 604p and a distal end 604d, and each of the proximal end 604p and the distal end 604d of the tubular adapter 604 are open. In one or more embodiments, the tubular adapter 604 comprises plastic material as described above. The tubular adapter comprises a proximal end 604p and a distal end 604d. In some embodiments, the tubular adapter 604 is transparent.

In one or more embodiments, the medical filtration assembly 105 further comprises a needle hub 608 including a distal end 608d and proximal end 608p. In some embodiments, the needle hub 608 comprises plastic. In the embodiment shown, the needle hub 608 is a needle hub is configured to receive a needle 609 which extends from the distal end 608d of the needle hub 608. The needle 609 includes a cannula 609c and may be made of metal and have a distal tip 609d.

In some embodiments, the needle hub 608 is attached to the medical filtration assembly 105 by inserting the distal end 608d of the needle hub 608 through the proximal end 604p of the tubular adapter 604. In one or more embodiments, the tubular adapter 604 and the needle hub 608 are assembled together by either press fitting or a snap fitting as described above.

In one or more embodiments, the distal end 604d of the tubular adapter 604, which is open, comprises a filter element 606. The tubular adapter 604 with the open distal end 604d and the filter element 606 is configured to aspirate medication through the filter element 606. In some embodiments, the filter element 606 is assembled inside the distal end 604d of the tubular adapter 604. In some embodiments the filter element 606 is sealed inside the distal end 604d of the tubular adapter 604 by a sealing method, for example insert molding, adhesive bonding, ultrasonic welding, or by any other suitable sealing method. In this way, the filter element 606 is integrated with the medical filtration assembly 105. In some embodiments, the filter element 606 has a porosity configured to filter glass particles or glass shards that are present inside a glass ampoule.

The medical filtration assembly 105 further comprises a shield 600, which is removable and has a distal end 600d and a proximal end 600p. In some embodiments, the distal end 600d of the shield 600 is flat and blunt. In other embodiments, the distal end 600d of the shield 600 is closed. In some embodiments, the shield 600 covers the distal end 604d of the tubular adapter 604. In some embodiments, the shield 600 covers a portion of the needle hub 608. In some embodiments, the shield 600 does not cover the needle hub 608. In some embodiments, the shield 600 is transparent. The shield 600 provides protection from foreign material entering the medical filtration assembly prior to its use.

In one or more embodiments, sealing between the proximal end 604p of the tubular adapter 604 and the needle hub 608 is a plastic-to-plastic or plastic-to-rubber interaction. The tubular adapter 604 according to one or more embodiments provides an open cover pressed over a needle hub 608 but having an opening in distal end 604d for aspirating drug into needle through filter element 606. In some embodiments, the tubular adapter 604 is extruded, and the tubular adapter 604 and the shield 600 seat directly on the needle hub 608 to that the proximal end 600p of the removable shield 600 is slidably engaged with the needle hub 608 to secure the removable shield 600 to the medical filtration assembly 105. The proximal end 600p of the shield 600 covers a portion of the proximal end 608p of the needle hub 608.

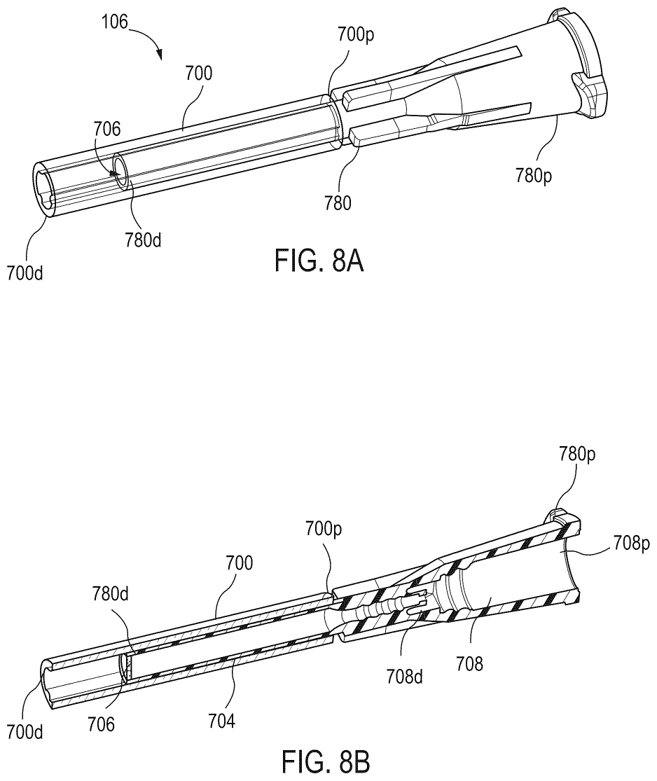

In a sixth embodiment of a medical filtration assembly 106, shown in FIGS. 8A-B, a tubular adapter 704 extends from the distal end 708d of the needle hub 708. In some embodiments, the tubular adapter 704 and the needle hub 708 are separate components joined together by snap fitting, press fitting or by adhesive or plastic welding. In some embodiments, the tubular adapter 704 is integrally formed with the needle hub to provide a combined needle hub/adapter 780. In some embodiments, the medical filtration assembly shown in FIGS. 8A-B may be referred to as a needle-less assembly.

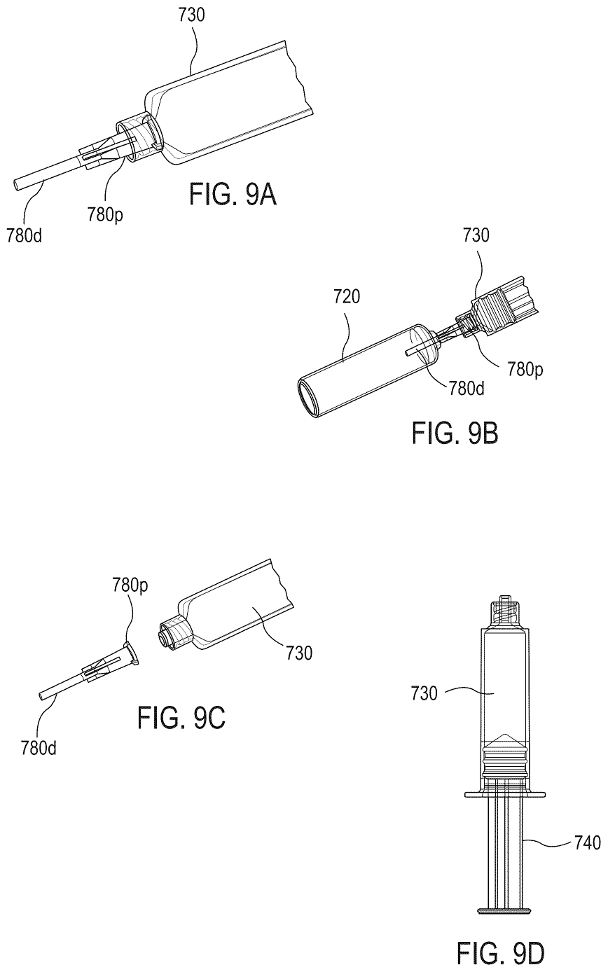

FIGS. 8A-B show the medical filtration assembly 106 configured for use with a medical device and configured to transfer filtered medication from a container for delivery to an IV bag or to a needle free connector mounted to patient, the medical filtration assembly 106, including the combined needle hub/adapter 780. The medical filtration assembly 106 shown in FIGS. 8A-B is configured to be attached to a medical device such as a syringe 730 including a plunger rod 740 as shown in FIGS. 9A-D. The medical filtration assembly 106 is configured so that a syringe 730 attached to the medical filtration assembly 106 is configured to aspirate medication from a glass ampoule 720 and injected to an IV bag (not shown).

The medical filtration assembly 106 shown comprises a tubular adapter 704 that is open ended. In one or more embodiments, the tubular adapter 704 comprises plastic material as described above. In some embodiments, the tubular adapter 704 is transparent.

In one or more embodiments, the medical filtration assembly 106 further comprises a needle hub 708 including a distal end 708d and proximal end 708p. In some embodiments, the needle hub 708 comprises plastic.

In one or more embodiments, the distal end 780d of the combined needle hub/adapter 780, which is open, comprises a filter element 706. The combined needle hub/adapter 780 with the open distal end 780d and the filter element 706 are configured to aspirate medication through the filter element 706. In some embodiments, the filter element 706 is assembled inside the distal end 780d of the combined needle hub/adapter 780. In some embodiments the filter element 706 is sealed inside the distal end 780d of the combined needle hub/adapter 780 by a sealing method, for example insert molding, adhesive bonding, ultrasonic welding, or by any other suitable sealing method. In this way, the filter element 706 is integrated with the medical filtration assembly 106. In some embodiments, the filter element 706 has a porosity configured to filter glass particles or glass shards that are present inside a glass ampoule.

The medical filtration assembly 106 further comprises a shield 700, which is removable and has a distal end 700d and a proximal end 700p. In some embodiments, the distal end 700d of the shield 700 is flat and blunt. In other embodiments, the distal end 700d of the shield 700 is closed. In some embodiments, the shield 700 covers the distal end 780d of combined needle hub/adapter 780. In some embodiments, the shield 700 covers a portion of the needle hub 708. In some embodiments, the shield 700 does not cover the needle hub 708. In some embodiments, the shield 700 is transparent. The shield 700 provides protection from foreign material entering the medical filtration assembly prior to its use.

FIGS. 8A-B show the medical filtration assembly 106 configured for use with a medical device and configured to transfer filtered medication from a container for delivery to an IV bag. The medical filtration assembly 106 shown in FIGS. 8A-B is configured to be attached to a medical device such as a syringe as shown in the first two embodiments. The medical filtration assembly 106 is configured so that a syringe (not shown) attached to the medical filtration assembly 106 is configured to aspirate medication from a glass ampoule (not shown) and injected to an IV bag.

FIGS. 9A-D illustrates use of the medical filtration assembly 106 shown in FIGS. 8A-B. FIG. 9A illustrates the medical filtration assembly 106 after a glass ampoule 720 has been broken open and the shield 700 has been removed. FIG. 9B shows inserting the combined needle hub/adapter 780 of the medical filtration assembly 106 into the glass ampoule 720 to aspirate medication from the ampoule 720 into the syringe 730. FIG. 9C illustrates removal of the combined needle hub/adapter 780 from medical filtration assembly. FIG. 9D illustrates the syringe 730 in a vertical position to remove air from the syringe 730 and ready for injection to a patient connector.

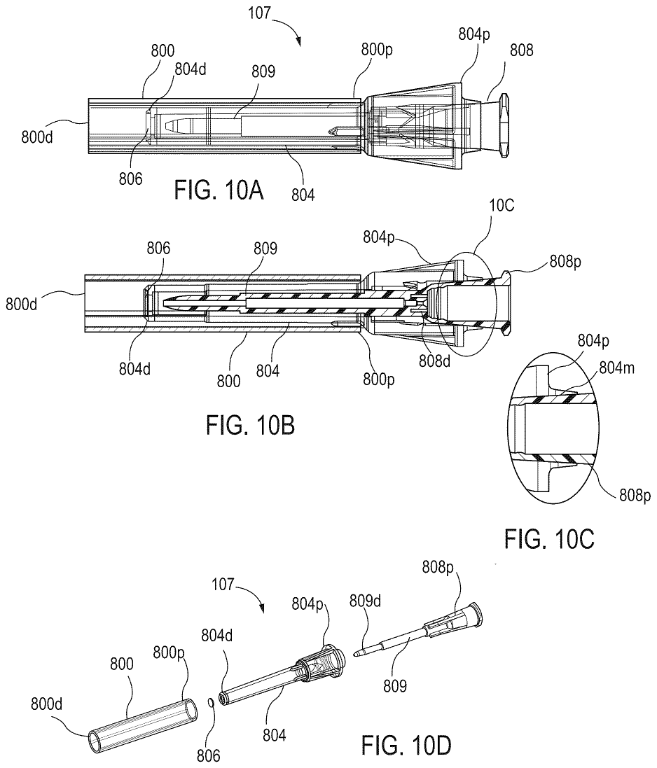

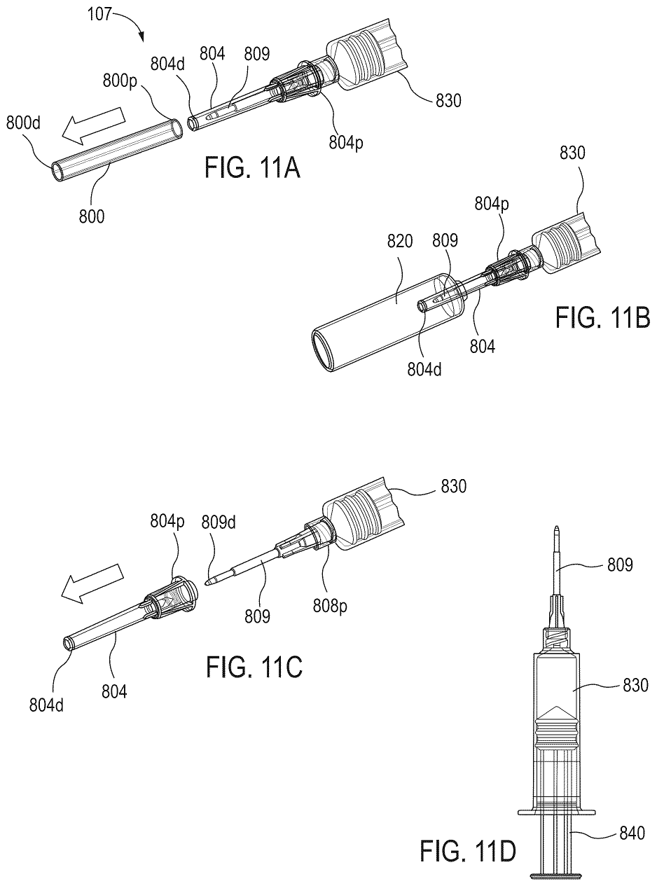

In a seventh embodiment of a medical filtration assembly 107, shown in FIGS. 10A-D, a plastic cannula 809 having a distal end 809d and proximal end 809p which extends from the distal end 808d of the needle hub 808. FIGS. 10A-D show the medical filtration assembly 107 configured for use with a medical device and configured to transfer filtered medication from a container for delivery to an IV bag using the plastic cannula 809. The medical filtration assembly 107 shown in FIGS. 10A-D is configured to be attached to a medical device such as a syringe 830 including a plunger rod 840 as shown in FIGS. 11A-D. The medical filtration assembly 107 is configured so that a syringe 830 attached to the medical filtration assembly 107 is configured to aspirate medication from a glass ampoule 820 and injected to an IV bag (not shown).

The medical filtration assembly 107 shown comprises a tubular adapter 804 that is open ended. In one or more embodiments, the tubular adapter 804 comprises plastic material as described above. In some embodiments, the tubular adapter 804 is transparent. In one or more embodiments, the medical filtration assembly 107 further comprises a needle hub 808 including a distal end 808d and proximal end 808p. In some embodiments, the needle hub 808 comprises plastic. In one or more embodiments, the tubular adapter 804 and the needle hub 808 are assembled together by an interference fit shown in detail in FIG. 10C, showing the mating surface 804m of the adapter with the needle hub 808. In some embodiments, the interference fit achieves an airtight seal through plastic-to-plastic contact between the tubular adapter 804 and the needle hub 808. In some embodiments, the needle hub 808 is a plastic hub with the plastic cannula 809, for example, a plastic needle such as a BD Interlink™ blunt plastic cannula having a blunt distal end 809d, without requiring metal needle to penetrate an IV bag or a medical device septum such as an IV tubing septum.

In one or more embodiments, the distal end 804d of the tubular adapter 804, which is open, comprises a filter element 806. The tubular adapter 804 with the open distal end 804d and the filter element 806 are configured to aspirate medication through the filter element 806. In some embodiments, the filter element 806 is assembled inside the distal end 804d of the tubular adapter 804. In some embodiments the filter element 806 is sealed inside the distal end 804d of the tubular adapter 804 by a sealing method, for example insert molding, adhesive bonding, ultrasonic welding, or by any other suitable sealing method. In this way, the filter element 806 is integrated with the medical filtration assembly 107. In some embodiments, the filter element 806 has a porosity configured to filter glass particles or glass shards that are present inside a glass ampoule.

The medical filtration assembly 107 further comprises a shield 800, which is removable and has a distal end 800d and a proximal end 800p. In some embodiments, the distal end 800d of the shield 800 is flat and blunt. In other embodiments, the distal end 800d of the shield 800 is closed. In some embodiments, the shield 800 covers the distal end 804d of the tubular adapter 804. In some embodiments, the shield 800 covers a portion of the needle hub 808. In some embodiments, the shield 800 does not cover the needle hub 808. In some embodiments, the shield 800 is transparent. The shield 800 provides protection from foreign material entering the medical filtration assembly prior to its use.

FIGS. 10A-D show the medical filtration assembly 107 configured for use with a medical device and configured to transfer filtered medication from a container for delivery to an IV bag. The medical filtration assembly 107 shown in FIGS. 10A-D is configured to be attached to a medical device such as a syringe as shown in the first two embodiments. The medical filtration assembly 107 is configured so that a syringe 830 attached to the medical filtration assembly 107 is configured to aspirate medication from a glass ampoule 820 and injected to an IV bag.

FIGS. 11A-D illustrates use of the medical filtration assembly 107 shown in FIGS. 10A-D. FIG. 11A illustrates removal of the shield 800 from the medical filtration assembly 107 attached to a syringe 830 by sliding the shield in the direction of the hollow arrow. FIG. 11B shows inserting the plastic cannula 809 with the blunt distal end 809d of the medical filtration assembly 107 into the glass ampoule 820 to aspirate medication from the ampoule 820 into the syringe 830. FIG. 11C illustrates removal of the tubular adapter 804 from the assembly. FIG. 11D illustrates the syringe 830 with the medical filtration in a vertical position to remove air from the syringe 830 and ready for injection into an IV bag, to a needle less connector or IV tubing septum.

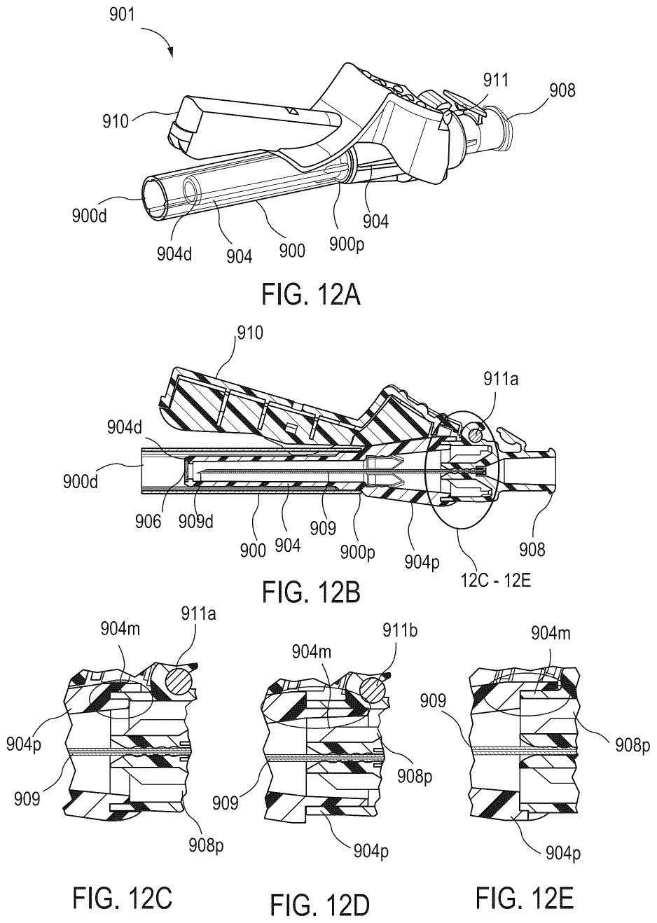

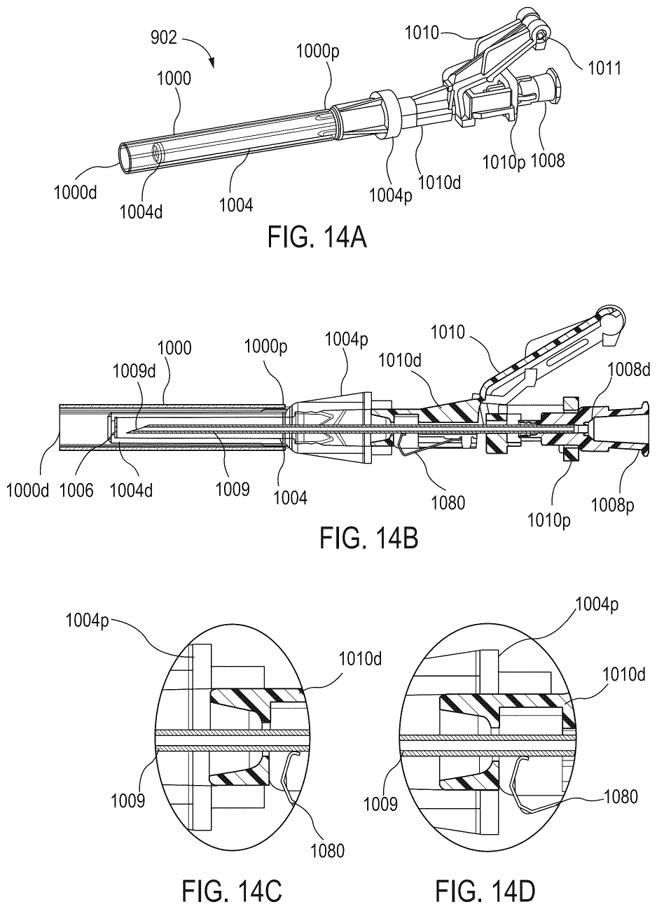

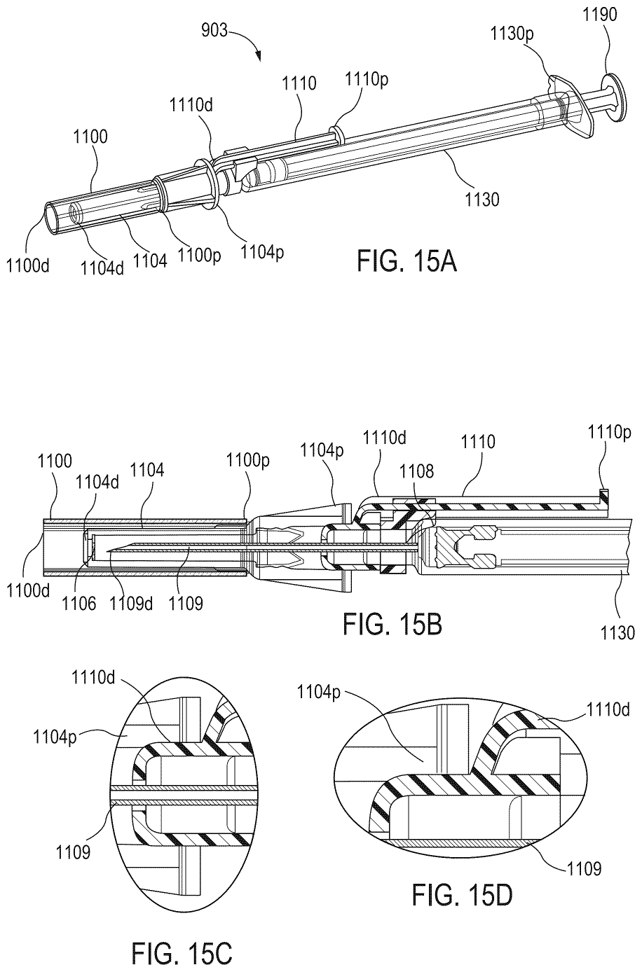

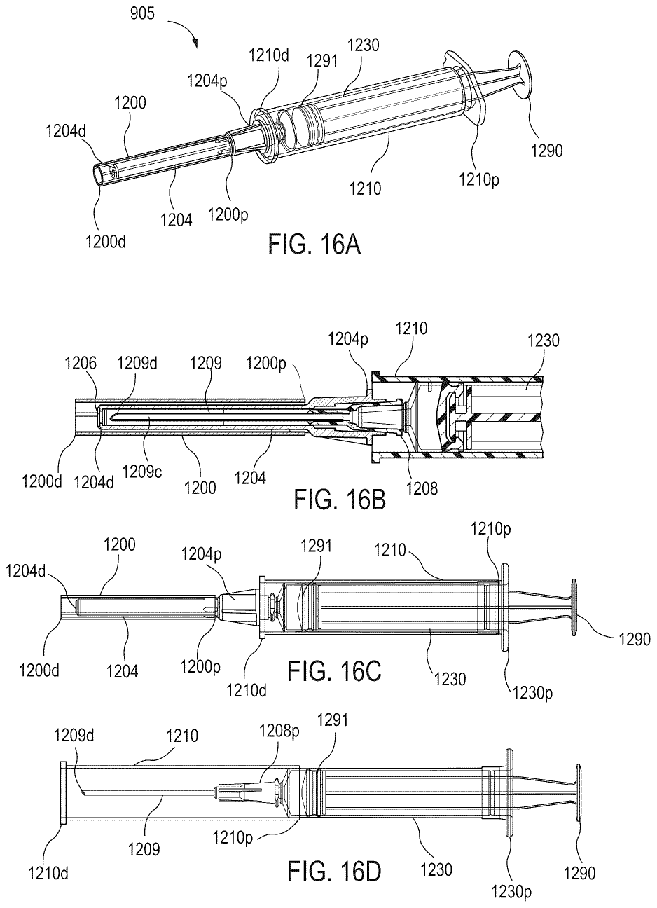

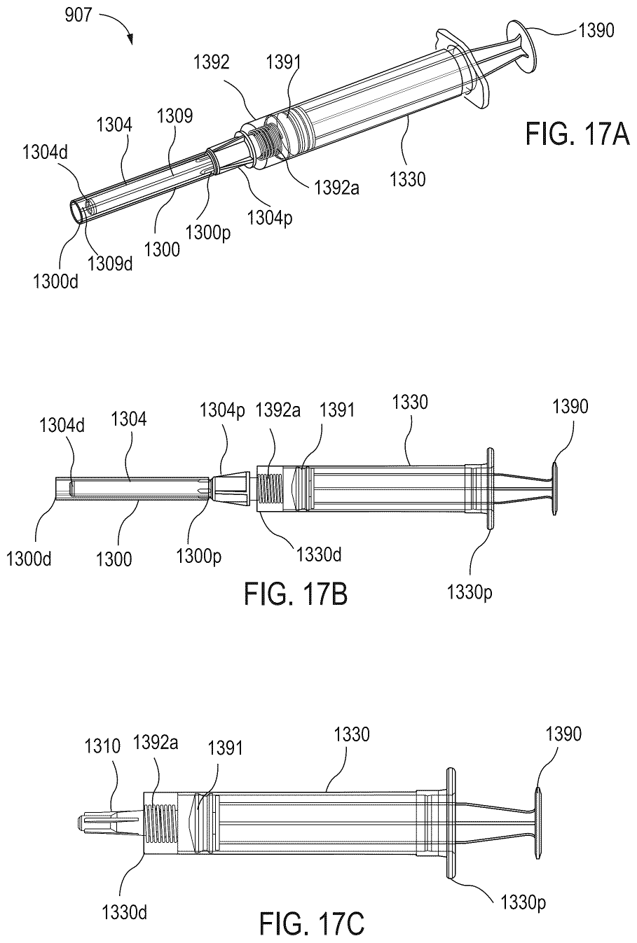

In one or more embodiments, the medical filtration assembly further comprises a safety shield. FIGS. 12A-E, FIGS. 14A-D, FIGS. 15A-D, FIGS. 16A-D and FIGS. 17A-C show different embodiments of medical filtration assemblies with different types of safety shields. There is a need for medical filtration assemblies configured to both filter medication from glass ampoules to streamline the medication filtration process as well as to reduce the risk of needle sticks for care providers.

A first embodiment of a medical filtration assembly 901 including a safety shield 910 is shown in FIGS. 12A-E. The medical filtration assembly 901 shown includes a needle hub 908, a tubular adapter 904 having a proximal end 904p and a distal end 904d with a filter 906 at the distal end, similar to the embodiments described above. A needle 909, for example, a hypodermic needle, with a distal tip 909d extends from the needle hub 908 having a proximal end 908p. In the embodiment shown, the safety shield comprises a hinged safety shield 910 including a hinge 911 having a first hinge element 911a engaged with a second hinge element 911b on the needle hub 908, the safety shield configured to pivot about the hinge to cover the distal tip 909d of the needle 909. In one embodiment the first hinge element 911a is a hinge pin extending from the safety shield 910, and the second hinge element 911b is a hinge knuckle configured to receive the first hinge element 911a. A shield 900 having a distal end 900d and a proximal end 900p covers the tubular adapter 904 when the medical filtration assembly 901 is not in use.

FIGS. 12C-E show details of the hinge 911 and the interaction of the needle hub 908 and the tubular adapter 904. There is a mating surface 904m where the needle hub 908 and distal end 904d of the tubular adapter 904 are sealed together.

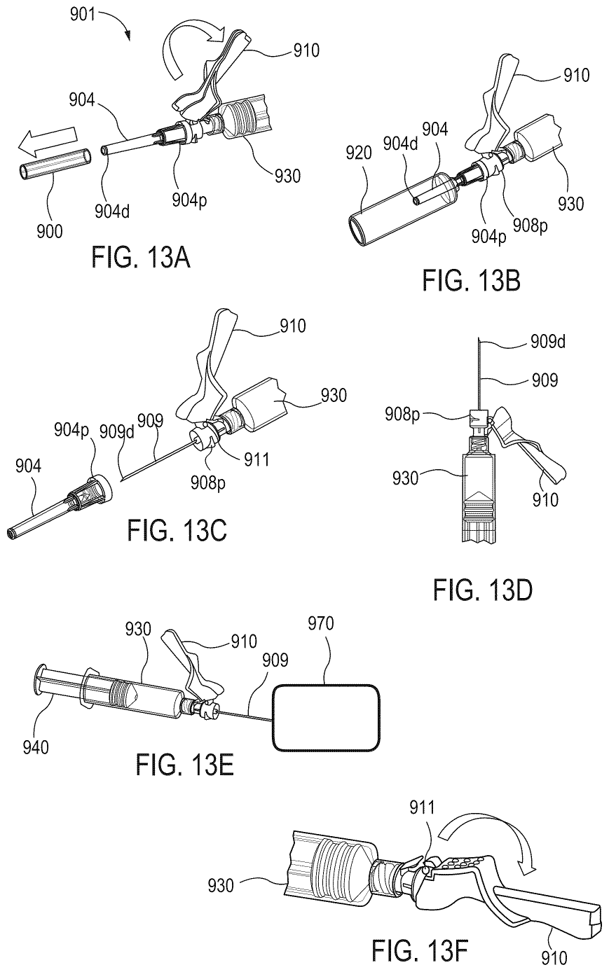

FIGS. 13A-F illustrate use of the medical filtration assembly 901 for aspiration from a glass ampoule with the medical filtration assembly 901 attached to a syringe. FIG. 13A shows the safety shield 910 rotated back about the hinge 911 and the shield 900 is removed. FIG. 13 shows medication being withdrawn from the glass ampoule 920 via the tubular adapter 904 and into the syringe, with the safety shield 910 in the open position. In FIG. 13C, the tubular adapter 904 is removed to expose the needle, and the safety shield 910 is still in the open position. The syringe 930 including a plunger rod 940 is positioned vertically to remove air from the medication as shown in FIG. 13D, with the safety shield 910 still in the open position. In FIG. 13E, medication is injected from the syringe into a patient 970, with the safety shield 910 still in the open position. After injection has been completed, a practitioner activates the safety shield 910 by manually pivoting the safety shield 910 about the hinge 911 until the safety shield 910 covers the distal tip 909d of the needle. In some embodiments, the safety shield is integral with the needle hub 908, or the safety shield 910 be a separate component.

A second embodiment of a medical filtration assembly 902 including a safety shield 1010 is shown in FIGS. 14A-D. The medical filtration assembly 902 shown includes a needle hub 1008, a tubular adapter 1004 having a proximal end 1004p and a distal end 1004d with a filter 1006 at the distal end, similar to the embodiments described above. A needle 1009, for example, a hypodermic needle, with a distal tip 1009d extends from the needle hub 1008 having a proximal end 1008p. In the embodiment shown, a needle having the safety shield comprises a slidable safety shield 1010 including a hinge 1011. The safety shield 1010 includes a mounting element 1010p engaged with the needle hub 1008 and a shield element 1010d that slides in a distal direction to cover a distal tip 1009d of the needle 1009. The hinge 1011 is configured to permit the shield element to extend over the distal tip of the needle. The safety shield 1010 may further comprise a clip 1080 that may lock over the distal tip 1009d of the needle 1009 when the safety shield 1010 is in a closed position. A shield 1000 having a distal end 1000d and a proximal end 1000p covers the tubular adapter 1004 when medical filtration assembly 902 is not in use.

FIGS. 14C-D show interaction of the shield element 1010d and the proximal end 1004p of tubular adapter 1004. The shield element 1010d in some embodiments is sealed to the distal end 1008d of the needle hub 1008.

A third embodiment of a medical filtration assembly 903 including a safety shield 1110 is shown in FIGS. 15A-D. The medical filtration assembly 903 shown includes a needle hub 1108, a tubular adapter 1104 having a proximal end 1104p and a distal end 1104d with a filter 1106 at the distal end, similar to the embodiments described above. A needle 1109, for example, a hypodermic needle, with a distal tip 1109d extends from the needle hub 1108 having a proximal end 1108p. In the embodiment shown, the safety shield comprises a slidable safety shield 1110 having a distal tab 1110p. Upon application of a distally directed force on the distal tab 1110p, the slidable safety shield 1010 slides in a distal direction to cover a distal tip 1109d of the needle 1109. The slidable safety shield 1110 is slidably mounted over the syringe 1130 including a plunger 1190. A shield 1100 having a distal end 1100d and a proximal end 1100p covers the tubular adapter 1104 when the medical filtration assembly 903 is not in use.

FIGS. 15C-D show interaction of the slidable safety shield 1110 and the proximal end 1104p of tubular adapter 1104. To use the device for aspiration, the shield 1100 is removed, and medication aspirated from an ampoule into a syringe connected to the medical filtration assembly 903. Once the medication is injected to patient, the slidable safety shield 1110 is activated manually by sliding it over the distal tip 1109d of the needle 1109.

A fourth embodiment of a medical filtration assembly 905 including a safety shield is shown in FIGS. 16A-D. The medical filtration assembly 905 shown includes a needle hub 1208, a tubular adapter 1104 having a proximal end 1204p and a distal end 1204d with a filter 1206 at the distal end 1204d, similar to the embodiments described above. A needle 1209, for example, a hypodermic needle, with a distal tip 1209d extends from the needle hub 1208. In the embodiment shown, the safety shield comprises a slidable safety shield 1210 comprising a slidable cover having a proximal end 1210p and a distal end 1210d that is slidably mountable over and surrounds a syringe 1230 and configured to cover a distal tip 1209d of the needle 1209. A shield 1200 having a distal end 1200d and a proximal end 1200p covers the tubular adapter 1204 when the medical filtration assembly 905 is not in use.

FIGS. 16C-D show activation of the slidable safety shield 1210 having the distal end 1210d before activation to cover the distal tip 1209d of the needle 1209. After aspiration of medication from and ampoule into the syringe and injection of the medication into a patient, the slidable safety shield 1210 that surrounds the syringe 1230 is activated manually by sliding it over the distal tip 1209d of the needle 1209 by applying a distally directed force to the proximal end 1210p of the slidable safety shield 1210b. The syringe 1230 includes a plunger rod 1290 and a stopper 1291.

A fifth embodiment of a medical filtration assembly 907 including a safety shield 1010 is shown in FIGS. 17A-C. The medical filtration assembly 907 shown includes a needle hub (not shown) with a needle 1309 having a distal tip 1309d extending from the needle hub. A tubular adapter 1304 having a proximal end 1304p and a distal end 1304d has a filter (not shown), similar to the embodiments described above. A needle 1309, for example, a hypodermic needle, with a distal tip 1309d extends from the needle hub. In the embodiment shown, a passive safety mechanism 1392 configured to be activated by a spring element 1392a retracts the needle behind the safety shield 1310 to cover the distal tip 1309d of the needle 1309. A shield 1300 having a distal end 1300d and a proximal end 1300p covers the tubular adapter 1304 when the medical filtration assembly 907 is not in use.

The passive safety mechanism 1392 according to one or more embodiments is configured similar to BD Integra™ Syringe with a spring-biased retracting needle c. In use, the shield 1300 is removed, medication is aspirated from a glass ampoule into a syringe 1330 including a plunger 1390 and a stopper 1391. The medication is injected into a patient form the syringe, and the passive safety mechanism 1392 is activated passively by pushing the plunger 1390, which activates the spring element 1392a to withdraw the needle 1309.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure includes modifications and variations that are within the scope of the appended claims and their equivalents.

What is claimed is:

1. A medical filtration assembly configured for use with a medical device, the medical filtration assembly comprising:
a tubular adapter comprising a proximal end and a distal end including an opening;
a filter element on the distal end of the tubular adapter and covering the opening;
a needle hub comprising a proximal end and a distal end, the proximal end of the of the tubular adapter configured to cover the distal end of the needle hub; and
a removable shield comprising an open proximal end and a distal end extending past the distal end of the tubular adapter, the removable shield sized and configured to cover a portion of the tubular adapter including the distal end of the tubular adapter.

2. The medical filtration assembly of claim 1, wherein the filter element is configured to filter particles from a liquid medium.

3. The medical filtration assembly of claim 1, wherein the tubular adapter is made from a polymeric material and the filter element is made from a porous polymeric material.

4. The medical filtration assembly of claim 3, wherein the filter element is bonded to the distal end of the tubular adapter.

5. The medical filtration assembly of claim 4, wherein the filter element is press-fitted to the distal end of the tubular adapter.

6. The medical filtration assembly of claim 2, wherein the removable shield is transparent.

7. The medical filtration assembly of claim 2, further comprising a blunt fill needle extending from the needle hub.

8. The medical filtration assembly of claim 2, further comprising a hypodermic needle comprising a sharp distal tip extending from the needle hub.

9. The medical filtration assembly of claim 2, further comprising a plastic cannula extending from the needle hub.

10. The medical filtration assembly of claim 2, wherein the tubular adapter has a first snap fitting element that engages the needle hub.

11. The medical filtration assembly of claim 2, further comprising a washer fitted to distal end of the tubular adapter and in contact with the filter element to secure the filter element to the tubular adapter.

12. The medical filtration assembly of claim 2, wherein the proximal end of the removable shield is slidably engaged with the tubular adapter to secure the removable shield to the medical filtration assembly.

13. The medical filtration assembly of claim 2, wherein the proximal end of the removable shield is slidably engaged with the needle hub to secure the removable shield to the medical filtration assembly.

14. The medical filtration assembly of claim 2, wherein the tubular adapter extends from the distal end of the needle hub.

15. The medical filtration assembly of claim 14, wherein the tubular adapter is integrally formed with the needle hub.

16. The medical filtration assembly of claim 1, further comprising a safety shield configured to cover a distal end of a needle.

17. The medical filtration assembly of claim 16, further comprising a needle having a distal tip extending from the needle hub, wherein the safety shield comprises a hinged safety shield including a hinge having a first hinge element engaged with a second hinge element on the needle hub, the safety shield configured to pivot about the hinge to cover the distal tip of the needle.

18. The medical filtration assembly of claim 16, further comprising a needle having a distal tip extending from the needle hub, wherein the safety shield comprises a slidable safety shield including a mounting element engaged with the needle hub and a shield element that slides in a distal direction to cover a distal tip of the needle.

19. The medical filtration assembly of claim 18, wherein the safety shield further comprises a hinge connecting the shield element and the mounting element, which is configured to permit the shield element to extend over the distal tip of the needle.

20. The medical filtration assembly of claim 16, further comprising a needle having a distal tip extending from the needle hub, wherein the safety shield comprises a slidable shield activated by applying a distally directed force to slide the safety shield over the distal tip of the needle.

21. The medical filtration assembly of claim 16, further comprising a needle extending from the needle hub, wherein the safety shield comprises a slidable cover that is slidably mountable over a syringe barrel and surrounds the syringe barrel and is configured to cover a distal tip of the needle.

22. The medical filtration assembly of claim 16, further comprising a needle extending from the needle hub, wherein the safety shield comprises a passive safety mechanism configured to be activated by a spring element to advance the safety shield to cover a distal tip of the needle.
